(19) United States
(12) Patent Application Publication
(10) Pub. No.: US 2020/0232989 A1
(43) Pub. Date: Jul. 23, 2020

(54) METHODS AND KITS FOR PREDICTING THE SENSITIVITY OF A SUBJECT SUFFERING OF RENAL CANCER TO SUNITINIB

(71) Applicants: UNIVERSITE D'AIX-MARSEILLE, MARSEILLE (FR); UNIVERSITE COTE D'AZUR, NICE CEDEX 2 (FR); ASSISTANCE PUBLIQUE HÔPITAUX DE MARSEILLE, MARSEILLE CEDEX 5 (FR); CENTRE NATIONAL DE LA RECHERCHE SCIENTIFIQUE, PARIS (FR); INSERM (INSTITUT NATIONAL DE LA SANTÉ ET DE LA RECHERCHE MÉDICALE), PARIS (FR)

(72) Inventors: MARCEL BLOT-CHABAUD, FUVEAU (FR); GILLES PAGES, MONACO (MC); MAEVA DUFIES, NICE (FR); NATHALIE BARDIN, MARSEILLE (FR); FRANÇOISE DIGNAT-GEORGE, MARSEILLE (FR)

(21) Appl. No.: 16/632,402

(22) PCT Filed: Jul. 20, 2018
(86) PCT No.: PCT/EP2018/069835

§ 371 Date: Jan. 20, 2020

(30) Foreign Application Priority Data
Jul. 21, 2017 (EP) 17305972.6

(51) Int. Cl. G01N 33/574

(52) U.S. Cl. CPC G01N 33/57438; G01N 2333/70596; G01N 33/57488

(57) ABSTRACT

The present invention relates to a method of predicting or monitoring the sensitivity of a subject having a cancer, in particular renal cell carcinoma (RCC) to sunitinib, to a method of selecting an appropriate treatment of cancer, to a method of screening or identifying a compound suitable for improving the treatment of a cancer, and to corresponding kits.

### FIELD OF THE INVENTION

The present invention relates to a method for predicting or monitoring the sensitivity of a subject having a tumor, to cancer treatment (also herein identified as “chemotherapy”), in particular a method of predicting or monitoring the sensitivity of a subject having renal cell, typically renal cancer carcinoma (RCC), preferably a metastatic clear cell renal cell carcinoma (ccRCC), to sunitinib, to a method of selecting an appropriate treatment of cancer, to a method of screening or identifying a compound suitable for improving the treatment of a cancer, and to corresponding kits and uses thereof. The method of predicting or monitoring the sensitivity of a subject having renal cancer to sunitinib typically comprises a step a) of determining, in a biological sample from said subject, the expression level of soluble CD146 (sCD146), and when the expression level is determined, a step b) of comparing said expression level to a sCD146 reference expression level, thereby assessing or monitoring whether the subject having renal cancer is responsive or resistant to sunitinib.

### BACKGROUND OF THE INVENTION

Renal cell carcinoma (RCC, also known as hypernephroma) is a renal cancer that originates in the lining of the proximal convoluted tubule, the very small tubes in the kidney that filter the blood and remove waste products. Clear cell metastatic renal cell carcinoma (ccRCC) is the most common type of kidney cancer in adults, responsible for approximately 80% of cases (Escudier et al, 2013; Mulders, 2008). It is the most lethal of all the genitourinary tumors.

Metastatic clear cell renal cell carcinomas (ccRCC) are highly angiogenic tumors bearing a mutation, deletion or methylation of the pvhl gene that leads to over-expression of VEGF. For this reason, anti-angiogenic treatments targeting the VEGF/VEGFR pathway were developed. However, these treatments ineluctably lead to therapeutic escape and disease progression, especially for the reference treatment, sunitinib, a multi kinase inhibitor of VEGF, PDGF, CSF1 receptors, c-KIT, FLT3 and RET (Choueiri et al, 2008). At relapse on sunitinib, other TKIs or mTOR inhibitors are available for second or third line including axitinib (Motzer et al, 2013a), pazopanib (Motzer et al, 2013b), cabozantinib (Choueiri et al, 2015), lenvatinib (Motzer et al, 2016) and everolimus (Motzer et al, 2008) Immunotherapies have also demonstrated promising results in recent clinical trials (Escudier et al, 2017).

Of interest, the response of patients to sunitinib is very heterogeneous. Some patients are refractory from the beginning and die rapidly. Most patients have a transient response and then relapse. Unfortunately, only a minority of patients are responders for a very long period of time (Gore et al, 2015). This suggests that RCC constitutes a heterogeneous tumor with variable responses to the anti-angiogenic treatment.

The heterogeneity of response to sunitinib, the current therapeutic standard, is a real concern. Identifying patients likely to respond to treatment represents a therapeutic challenge to limit the administration of an ineffective toxic product and rapidly adapt the treatment. The discovery of a predictive marker of response to sunitinib and accurate selection of patients capable of responding to this particular chemotherapy is a solution for patients to receive the most appropriate therapy as soon as possible and avoid a possibly toxic one.

### SUMMARY OF THE INVENTION

The present invention is based on the discovery by inventors that soluble CD146 (sCD146) is a predictive marker of response to a sunitinib treatment of renal cancer, more specifically renal cell carcinoma (RCC), in particular clear cell renal cell carcinoma (ccRCC) or metastatic ccRCC. Thus, the present invention includes methods and kits for assessing, typically for determining, for predicting or for monitoring, the response of a subject having a renal tumor or cancer to a particular chemotherapeutic treatment (“chemotherapy”) involving sunitinib as sole active drug, using sCD146 as a predictive biomarker.

In the context of the present invention, the expressions “chemotherapeutic treatment of cancer” and “chemotherapy” generally designate a “cancer treatment” and for example cover any targeted cancer therapy such as those involving an inhibitor of a tyrosine kinase receptor, preferably sunitinib, or a (monoclonal) antibody.

A first method herein described is an in vitro or ex vivo method for assessing, typically for determining, for predicting or for monitoring, the sensitivity of a subject having a cancer or tumor to sunitinib, typically the sensitivity of a subject having a renal cancer, more specifically a renal cell carcinoma (RCC), in particular a clear cell renal cell carcinoma (ccRCC) or metastatic ccRCC, which method comprises a step of determining (typically measuring), in a biological sample from said subject, the expression level of sCD146 and a step of comparing said sCD146 expression level to a sCD146 reference expression level, thereby assessing whether the subject having a cancer or tumor, typically a renal cancer, is responsive or resistant to sunitinib.

A particular method herein described is a method, typically an in vitro or ex vivo method, of selecting an appropriate treatment of cancer, typically an appropriate chemotherapeutic treatment of cancer, for example a treatment comprising sunitinib, for a subject having a cancer, typically a renal cancer, more specifically a renal cell carcinoma (RCC), in particular a clear cell renal cell carcinoma (ccRCC) or metastatic ccRCC, which method comprises a step a) of determining (typically measuring) the sCD146 expression level in a biological sample of the subject having cancer before administration to the subject of sunitinib, typically before any administration to the subject of sunitinib, and a step b) of comparing said sCD146 expression level to sCD146 reference expression level, a sCD146 expression level identical to or above the sCD146 reference expression level, being the indication that sunitinib will not be efficient, or will not be efficient alone in the subject, and a step c) of selecting an appropriate treatment of cancer in the subject, for example combining sunitinib with an additional compound such as a sCD146 antibody; and, on the contrary, a sCD146 expression level below the sCD146 reference expression level being the indication that sunitinib will be efficient alone against cancer in the subject.

Also herein described is a method for screening or identifying a compound suitable for improving the treatment of a cancer, typically a renal cancer, as herein identified in a subject having such a cancer, said method comprising determining the ability of a test compound to modify the expression of sCD146, or compensate an abnormal expression thereof.

A further embodiment relates to the use of a kit i) for assessing or monitoring in vitro or ex vivo the sensitivity or resistance of a subject having a cancer, in particular a renal cancer, to a chemotherapy wherein sunitinib is used as sole (single) active drug, and/or ii) for determining in vitro or ex vivo the potential toxicity of said chemotherapy in a subject having a cancer, in particular a renal cancer, wherein the kit comprises detection means comprising at least one sCD146 binding agent, typically an antibody specific to sCD146; a molecule allowing the binding agent detection; and, optionally, a leaflet providing typically the reference expression level(s) in a control population.

### DETAILED DESCRIPTION OF THE INVENTION

CD146, also known as MCAM, MUC18, or Mel-CAM, is a component of the endothelial junction which belongs to the immunoglobulin superfamily (Bardin et al, 2001). As a member of such a family, it consists in five Ig domains, a transmembrane domain, and a cytoplasmic region. CD146 is mainly known to occur in two distinct forms differing by the length of their cytoplasmic domain: a long isoform (herein identified as “long CD146”—cf. human long CD146 corresponding to SEQ ID NO:1) and a short isoform (herein identified as “short CD146”—cf. human short CD146 corresponding to SEQ ID NO:2), both present in the membrane of cells, mainly endothelial cells.

CD146 was recently described as a new factor involved in tumor angiogenesis (Yan et al.). CD146 is indeed also neo-expressed in many tumors including lung, melanoma, pancreas, prostate, breast, stomach and renal tumors (Fenget al, 2012; Liu et al, 2012; Okaet al, 2012; Zeng et al, 2011)(Maitra et al, 2003; Wuet al, 2005)(Lehmann et al, 1989). In prostate cancer, it was shown that this neo-expression resulted from hypermethylation at the promoter of the CD146 gene (Liu et al, 2012). Of interest, CD146 was recently described as a co-receptor for VEGFR2 in tumor angiogenesis (Jiang et al, 2012). In addition to the membrane-anchored form of CD146, inventors recently identified a soluble form of CD146 (sCD146) which is generated by the shedding of the membrane form (Bardin et al, 1998; Bardin et al, 2003). This soluble form is produced by tumors expressing CD146 and displays both autocrine effects on proliferation and survival of cancer cells, and paracrine effects on tumor angiogenesis (Harhouri et al, 2010; Stalin et al, 2016). These effects are mediated through binding of sCD146 on the p80 isoform of angiomotin (Stalin et al, 2013) which is expressed on both endothelial (Roudieret al, 2009) and tumor cells (Jiang et al, 2012; Satchi-Fainaroet al, 2012) and was reported to inhibit YAP oncoprotein (Zhaoet al, 2011).

Inventors now advantageously herein reveal that sCD146 is an innovative tool which can be used as an early and non-invasive biomarker of cancer patient's response to sunitinib. Inventors also discovered that this property of sCD146 is specific to sunitinib as sCD146 is of no help to assess the sensitivity of a patient for example to bevacizumab and temserolimus or interferon alpha.

This soluble form of the CD146 protein, present in particular in the human serum, has been structurally and functionally characterized by inventors in patent application WO2010/086405 (also identified as EP2216399).

“Human soluble CD146 protein” or “soluble CD146” herein designates a human protein, peptide or amino acid molecule containing about 552 to about 558 amino acids, preferably 558 amino acids, even more preferably 557, 556, 555, 554, 553 or 552 amino acids. An example of a human soluble CD146 protein comprises at least residues 1 to 552 inclusive, preferably at least residues 1 to 557 inclusive, of the amino acid sequence SEQ ID NO: 1. A particular sCD146 protein comprises or consists in amino acid sequence SEQ ID NO: 3. Another particular sCD146 protein comprises or consists in amino acid sequence SEQ ID NO: 4, 5, 6, 7, 8 or 9. A preferred sCD146 amino acid sequence consists in SEQ ID NO:7. Also herein described are nucleic acid molecules, typically RNA or DNA, that preferably each encode a biologically active human CD146, in particular a human soluble CD146 of the invention, and recombinant forms thereof such as SEQ ID NO: 10, 11, 12, 13, 14, 15 and 16.

A first object herein described is thus an in vitro or ex vivo method of assessing, typically of determining, of predicting or of monitoring, the sensitivity of a subject having a cancer or tumor to a cancer treatment, typically a chemotherapeutic molecule, preferably sunitinib, which method comprises a step of determining, in a biological sample from said subject, the presence, absence or expression level, preferably the expression level, of sCD146, and typically a step of comparing said sCD146 expression level to a sCD146 reference expression level or reference value, thereby assessing whether the subject having a cancer or tumor is responsive or resistant to the cancer treatment.

The cancer or tumor is preferably a renal cancer.

A particular and preferred renal cancer is a renal cancer carcinoma, typically a clear cell renal cancer (ccRCC), in particular a metastatic clear cell renal cancer.

The renal cancer is conventionally treated with a chemotherapeutic drug, typically a cytokine (typically interferon alpha or interleukin 2); an anti-angiogenic drug for example selected from a tyrosine kinase inhibitor (in particular sunitinib), a serine kinase inhibitor (in particular a mTOR inhibitor, typically everolimus or temsirolimus) and a threonine kinase inhibitor; or a combination thereof, typically the combination of a cytokine and of an anti-angiogenic drug, preferably the combination of interferon alpha and bevacizumab. In a particular aspect, the renal cancer is treated with a tyrosine kinase inhibitor such as sunitinib, axitinib, pazopanib, sorafenib; the bevacizumab (VEGF trap also identified as aflibercept); or a serine kinase inhibitor (in particular a mTOR inhibitor, typically everolimus or temsirolimus).

In the context of the present invention, the patient or subject is a mammal In a preferred embodiment, the mammal is a human being, whatever its age or sex. The patient typically has a cancer or tumor. Unless otherwise specified in the present disclosure, the tumor is a cancerous or malignant tumor. In a preferred embodiment, the subject is i) a subject who has not been previously exposed to a treatment of cancer, in particular, when the cancer is a renal cancer, to a treatment of renal cancer as described herein above, or ii) a subject who has received at least a first administration of a treatment of cancer, typically of a chemotherapeutic drug as described herein above. In another preferred embodiment, the subject is a subject who has undergone at least partial resection of the cancerous tumor or a complete resection thereof, for example through a nephrectomy. Another particular subpopulation of subjects is composed of subjects having metastases, typically subjects suffering of a renal cancer having metastasis.

Implementations of the methods of the invention involve obtaining a (biological) sample from a subject. The sample is preferably a fluid sample and may be selected for example from blood, typically total blood, serum, plasma, urine, lymphatic fluid, spinal fluid, pleural effusion, ascites, and a combination thereof The sample is typically selected from a blood sample, a serum sample, a plasma sample, a urine sample or a derivative thereof. A particular sample is a plasma sample or a derivative thereof such as a plasma sample free of (or devoid of) blood platelets. Another preferred sample is a fluid sample without cells. The biological sample is preferably a diluted sample, the dilution being typically of 1/50, 1/100, 1/200 or 1/400.

By “sensitivity” or “responsiveness” is intended herein the likelihood that a patient will respond to a treatment of cancer, typically to a chemotherapeutic treatment thereof as herein described, preferably sunitinib.

By “resistant” is intended herein the likelihood that a patient will not respond to a treatment of cancer, typically to a chemotherapeutic treatment thereof as herein described, preferably sunitinib.

Predictive methods of the invention can be used clinically to make treatment decisions by choosing as soon as possible the most appropriate treatment modalities for a particular patient.

If the subject is identified, using a method according to the present invention, as resistant to a particular treatment of cancer, typically sunitinib, the method advantageously further comprises a step of selecting a distinct treatment of cancer, typically involving a “compensatory molecule”, to be used instead of or in combination with the originally preselected chemotherapeutic drug (typically sunitinib) or with a distinct chemotherapeutic drug, as the appropriate therapeutic treatment of cancer for the subject.

Preferably, the reference expression level of sCD146 is calculated from the expression level of sCD146 determined in a biological sample of the subject before any administration of a chemotherapeutic treatment in the subject, i.e. before any administration to the subject of a chemotherapeutic drug for treating the subject's cancer, preferably before any administration of sunitinib to the subject. Less preferably but also possible, this step can be performed after the first or a subsequent administration of a chemotherapeutic drug, preferably sunitinib to the subject. This step can be performed before any tumor surgical resection. It can also be performed on a subject who has undergone at least partial resection of the cancerous tumor or complete resection thereof.

In a preferred aspect, this step of determining the expression level of sCD146 in a biological sample of the subject is performed at least twice, a first time as described previously before any administration to the subject of a chemotherapy for treating the subject's cancer or after the first or a subsequent administration of such a chemotherapy, and at least a second time after either the first or subsequent (for example second or third) administration thereof in order to be able to compare the measured expression levels.

Inventors thus in particular herein describe an in vitro or ex vivo method of assessing, typically of determining, of predicting or of monitoring, the sensitivity of a subject having a cancer or tumor to sunitinib, which method comprises a step of determining, typically measuring, in a biological sample from said subject, the expression level of sCD146 after treatment of the subject with sunitinib, in particular after a first or subsequent treatment of the subject with sunitinib, or in a particular embodiment after a first or subsequent cycle of treatment with sunitinib, and typically a step of comparing said sCD146 expression level to a sCD146 reference expression level or reference value, thereby assessing whether the subject having a cancer or tumor is responsive or resistant to sunitinib.

In a particular aspect, the patient is a metastatic patient and the “first cycle of treatment with sunitinib” refers to a single daily administration of 50 mg sunitinib for one month (four weeks or 28 days), this cycle being typically followed by two weeks without treatment.

Typically, the “reference value” or “reference expression level” is the level of sCD146 in a control sample derived from one or more subjects (reference population) having a cancer, in particular a renal cancer, and is typically the median value when obtained from the reference population.

The measured sCD146 expression level, preferably the first measured sCD146 expression level, typically the sCD146 expression level measured at diagnosis of the tumor or cancer of the subject (before any cancer treatment of the subject, in particular chemotherapeutic treatment), is also typically usable to determine or calculate a sCD146 reference expression level which can then be compared to the measured sCD146 expression level.

In a preferred embodiment, the sCD146 reference expression level is calculated from the sCD146 expression level determined in a biological sample from the subject before any administration of sunitinib to said subject.

In a particular embodiment, the herein described in vitro or ex vivo method of assessing, typically of determining, of predicting or of monitoring, the sensitivity of a subject having a cancer or tumor to a cancer treatment, in particular sunitinib, comprises a step of determining, typically measuring, in a biological sample from said subject, the expression level of sCD146 after treatment of the subject with the cancer treatment, in particular after a first or subsequent treatment of the subject with the cancer treatment, or in a particular embodiment after a first or subsequent cycle of treatment with the cancer treatment, and typically a step of comparing said sCD146 expression level to a sCD146 reference expression level or reference value, thereby assessing whether the subject having a cancer or tumor is responsive or resistant to the cancer treatment, a sCD146 expression level identical to or above the sCD146 reference expression level, being the indication that the subject is resistant to the cancer treatment, in particular sunitinib, when used alone (i.e. as single active molecule); and, on the contrary, a sCD146 expression level below the sCD146 reference expression level being the indication that the subject is sensitive to the cancer treatment, in particular sunitinib, even when used alone.

In a particularly preferred embodiment where sunitinib is specifically used as the cancer treatment, the sCD146 reference expression level is equal to the sCD146 expression level, as determined in a biological sample from the subject before any administration of sunitinib to said subject, to which is added (plus) 120% of said sCD146 expression level (determined in the biological sample from the subject before any administration of sunitinib).

Inventors herein demonstrate that a sCD146 expression level below the sCD146 reference expression level is indicative of a sensitivity of the subject, typically of a subject suffering of a renal cancer as herein described, to sunitinib, and a sCD146 expression level identical to or above said reference expression level is indicative of a resistance of the subject, typically of a subject suffering of a renal cancer as herein described, to sunitinib.

Inventors also in particular herein describe an in vitro or ex vivo method of monitoring the sensitivity of a subject having a cancer or tumor to sunitinib, which method comprises a step of determining, typically measuring, in a biological sample from said subject, the expression level of sCD146 after treatment of the subject with sunitinib, and typically a step of comparing said sCD146 expression level to at least one sCD146 reference expression level or reference value as herein described, thereby assessing or monitoring whether the subject having a cancer or tumor is still responsive or has become resistant to sunitinib.

In such a monitoring method, the treatment of the subject with sunitinib is typically a second, third, or a subsequent treatment step with sunitinib.

In a particular embodiment, each measured sCD146 expression level is typically to be compared with the measure directly preceding the measure under consideration or with several of said preceding measures when existing.

In a preferred embodiment, each measured sCD146 expression level is typically to be compared with the measure performed before any treatment with, or any administration of, sunitinib.

In some embodiment the invention relates to a method for monitoring the treatment of a subject suffering from a cancer, typically a renal cancer, comprising i) determining the level of sCD146 in a sample obtained from the subject before the treatment or during the treatment, ii) determining the level of sCD146 in a sample obtained from the subject after the treatment or later during the treatment, iii) comparing the level determined at step i) with the level determined at step ii), and iv) concluding that treatment is effective when the level(s) determined at step ii) is below the level determined at step i) plus 120% thereof, or concluding that the treatment is not effective, or is not efficient enough, when the level(s) determined at step ii) is identical to or above the level determined at step i) plus 120% thereof.

When the treatment is identified as not effective or not efficient enough, a method including a step, as herein above described, of selecting an appropriate, preferably optimal, alternative therapeutic treatment of cancer will be advantageously performed.

Also herein described is indeed an vitro or ex vivo method of selecting, for a subject having a cancer, typically a renal cancer, more specifically a renal cell carcinoma (RCC), in particular a clear cell renal cell carcinoma (ccRCC) or metastatic ccRCC, a treatment (typically a chemotherapy) efficient against cancer in the subject, wherein the method comprises a step a) of determining, typically measuring, the sCD146 expression level in a biological sample of the subject having cancer preferably before administration, typically before any administration, to the subject of a cancer treatment, in particular of sunitinib, and a step b) of comparing said sCD146 expression level to sCD146 reference expression level, a sCD146 expression level identical to or above the sCD146 reference expression level, being the indication that the cancer treatment, in particular sunitinib, will not be efficient, or will not be efficient alone in the subject, and a step c) of selecting an appropriate, preferably optimal, treatment of cancer in the subject, for example combining sunitinib with an additional compound such as a sCD146 antibody; and, on the contrary, a sCD146 expression level below the sCD146 reference expression level being the indication that cancer treatment, in particular sunitinib, will be efficient alone against cancer in the subject, said cancer treatment being an appropriate treatment of the cancer.

In the methods herein described of assessing the sensitivity of a subject having a tumor to a cancer treatment as well as in the methods herein described of selecting an appropriate cancer treatment, any classical method known by the skilled person of determining the presence or measuring the expression level of a compound of interest, typically sCD146, can be performed.

Preferably, the determination of the presence as well as the measure of the quantities of sCD146, is determined in an immunoassay through a one step method wherein the subject's biological sample is directly contacted with the appropriate ligand or through a method implying a preliminary treatment of the biological sample. An immunoassay can typically be performed through well-known methods of the art: in solid phase or homogeneous phase, in one or two steps, through competitive method, etc. The immunoassay is typically selected from the group consisting of ELISA, FEIA, multiplex, western blot, dot blot, bead-based assay, antigen array and Radio Immuno Assay.

In an ELISA, an antigen must be immobilized to a solid surface and then complexed with an antibody that is linked to an enzyme. Detection is accomplished by assessing the conjugated enzyme activity via incubation with a substrate to produce a colored product.

In FEIA, the colored product is fluorescent.

In Radio Immuno Assay, the final product is radioactive.

Protein detection using the dot blot protocol is similar to western blotting in that both methods allow for the identification and analysis of proteins of interest. Dot blot methodology differs from traditional western blot techniques by not separating protein samples using electrophoresis. Sample proteins are instead spotted onto membranes and hybridized with an antibody probe. Bead-based assay or antigen array are new approaches for investigators to simultaneously measure multiple analytes in biological and environmental samples.

Semi-quantitative measurements can be obtained with each of the previously described methods using for example normal controls to normalize the value and then establish a ratio, or using a positive control as a calibrator (expressed in arbitrary units).

The detection may be performed on a solid support, for example a microplaque, or solid particles, test tubes, etc.

Preferably, the selected immunoassay is an ELISA or radioimmunoassay.

The present invention in particular covers methods involving using an ELISA assay to identify a sCD146 polypeptide. In some embodiments, the ELISA assay is a sandwich assay. In a sandwich assay, more than one antibody will be employed. Typically ELISA method can be used, wherein the wells of a microtiter plate are coated with a set of antibodies which recognize the protein of interest. A sample containing or suspected of containing the protein of interest is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

In some embodiments of the invention, identification of sCD146 involves use of at least one sCD146 binding agent, typically polypeptide binding agent. A preferred sCD146 binding agent is capable of selectively binding soluble CD146 (versus membrane CD146).

The sCD146 binding agent is, in particular embodiments, an antibody, for example a synthetic, monoclonal or polyclonal antibody, in particular an anti-sCD146 antibody capable of selectively binding soluble CD146 (versus membrane CD146).

Preferably, the anti-sCD146 antibody is selected from a specific anti-sCD146 antibody of the art. The antibody can be bi-specific, recognizing two different epitopes. The antibody, in some embodiments, immunologically binds to more than one epitope from the same sCD146 polypeptide.

A sCD146 polypeptide binding agent that is a polypeptide may also include all or part of a receptor for sCD146 polypeptides, such as angiomotin (Amot).

A particular sCD146 binding agent usable in the context of the invention is capable of binding both the human soluble CD146 protein and a CD146 or soluble CD146 protein receptor (or a CD146 protein receptor subunit).

The antibody binding sCD146 can be made by techniques well known in the art. Methods of making such antibodies are known in the art (see for example DespoixNb et al., 2008).

In preferred embodiments, the anti-sCD146 antibody is a monoclonal antibody. A monoclonal antibody selectively binding sCD146 (versus membrane CD146), usable in the context of the invention, is advantageously selected from clone COM 3D9, clone COM 2F6, clone COM 5G6, clone COM 7A4 and clone F4-35H7 (S-endo 1) from BioCytex, and is preferably clone COM 7A4.

Antibodies usable in the context of the present invention may also be anti-antibodies used to identify, follow, detect and/or measure the antibodies that recognize the sCD146.

In some embodiments of the invention, the soluble CD146 binding agent is an aptamer.

In some embodiments of the invention, the sCD146 binding agent, typically antibody or aptamer, is labelled with one or more tags (detection agent, marker or label) allowing for their identification, follow-up, detection and/or measurement. The detection agent, marker or label may be selected for example from a fluorophore, a chemiluminescent particle, a radioisotope, a magnetic bead, an antigenic epitope, an enzyme (such as horseradish peroxidase), a substrate of a specific enzyme, a ligand or binding domain of sCD146, and any other molecule or moiety which may be detected or quantified. A detection agent may be an antibody that binds to a sCD146 polypeptide binding agent, such as an antibody. The detection agent antibody, in some embodiments, binds to the Fc-region of a binding agent antibody.

It is also specifically contemplated that a binding agent is unlabelled, but may be used in conjunction with a detection agent that is labelled. A detection agent is a compound that allows for the detection or isolation of itself so as to allow detection of another compound that binds, directly or indirectly. An indirect binding refers to binding among compounds that do not bind each other directly but associate or are in a complex with each other because they bind the same compounds or compounds that bind each other.

Other embodiments of the invention involve a second sCD146 binding agent in addition to a first sCD146 binding agent. The second binding agent may be any of the entities discussed above with respect to the first binding agent, such as an antibody. It is contemplated that a second antibody may bind to the same or different epitopes as the first antibody. It is also contemplated that the second antibody may bind the first antibody or another epitope than the one recognized by the first antibody.

In a particular aspect herein described, the appropriate or optimal treatment of cancer as determined in the context of the previously described method comprises the administration of at least one anti-sCD146 antibody, such as any one of the herein described anti-sCD146 antibodies, preferably an anti-sCD146 monoclonal antibody. This(ese) antibody(ies) as well as any antibodies comprising CDR sequences of herein described antibodies or humanized versions thereof can be used as compensatory molecule(s) allowing the anti-cancer treatment to be effective in the subject.

The appropriate treatment of cancer as determined in the context of the previously described methods may, according to a distinct embodiment, comprise the administration of an inhibitor of a sCD146 receptor.

In a further distinct embodiment the appropriate treatment of cancer does not involve the initially envisioned cancer treatment, in particular sunitinib, which has been identified as inefficient alone in the subject thanks to the herein above described methods of the invention. In this embodiment, the appropriate treatment of cancer will preferably be a distinct cancer treatment (“alternative treatment”), typically selected from a cytokine, an anti-angiogenic drug, an inhibitor of a tyrosine kinase receptor, a mTOR inhibitor, an antibody as herein described, and any combination thereof.

In a preferred embodiment, when sunitinib has been identified thanks to present invention as inefficient alone in the subject, the appropriate treatment of cancer used as alternative treatment will be an inhibitor of a tyrosine kinase receptor as herein described typically selected from axitinib, pazopanib, cabozantinib, sorafenib and a combination thereof, preferably pazopanib, axitinib,cabozantinib and lenvatinib. This selected inhibitor of a tyrosine kinase receptor used in an alternative treatment can further be combined to an immune checkpoint inhibitor, typically selected from avelumab, nivolumab, ipilimumab, atezolizumab and a combination thereof, and/or to a further distinct inhibitor of a tyrosine kinase receptor. In a particular embodiment, the alternative molecule(s) and sunitinib are used successively, sunitinib being used first and the alternative molecule(s) being used after sunitinib.

In a preferred embodiment, when sunitinib has been identified thanks to present invention as inefficient alone in the subject, the appropriate treatment of cancer used as alternative and/or combined treatment will be a kinase inhibitor as herein above identified or a combination thereof, and/or an immune checkpoint inhibitor (cf. Motzer et al. and McDermott et al), typically selected from avelumab, nivolumab, pembrolizumab, ipilimumab, atezolizumab and a combination thereof.

Methods of screening for candidate therapeutic agents for preventing or treating cancer, in particular renal cancer, are also included as part of the invention. The method is typically a method which is performed in vitro or ex vivo. When performed in vitro or ex vivo, it can be performed for example on a sample from a subject who has been administered with a test compound.

A method herein described is typically a method for screening or identifying a candidate compound suitable for improving the treatment of a cancer, typically a renal cancer, as herein identified in a subject having such a cancer, said method comprising determining the ability of a test compound to modify the expression of sCD146, or compensate an abnormal expression thereof.

In a particular embodiment, the present invention relates to a method of identifying a candidate therapeutic agent, or screening for candidate therapeutic agents, for treating a cancer as herein described, typically a renal cancer, comprising i) providing one or a plurality of candidate compounds, ii) bringing the candidate compound(s) into contact with cancer cell lines, or cancer cells from the subject to be treated expressing CD146, in presence of agent(s) that modulate(s), or is/are tested for its/their ability to modulate, the expression of sCD146, iii) determining the level of sCD146 released by the cancer cell lines or cancer cells from the subject to be treated, iv) comparing the level determined at step iii) with the level determined in the absence of the candidate compounds, and v) positively selecting the candidate compounds when the level determined at step iii) is/are decreased when compared to the level determined in the absence of the candidate compounds.

In a particular aspect, the method is performed in the presence of sunitinib and the method allows the detection of candidate agent(s) capable of restoring sunitinib efficiency in cancer cells from a subject to be treated for a renal cancer herein identified, thanks to the present invention, as resistant to sunitinib when used as single active molecule.

The candidate compound may be a natural or synthetic compound.

The candidate compound can be any compound tested for its efficiency in the treatment of a carcinoma such as a renal cancer, in particular ccRCC, such as for example an anti-sCD146 monoclonal antibody.

The candidate compound according to the invention may be selected from a library of compounds previously synthesised, or a library of compounds for which the structure is determined in a database, or from a library of compounds that have been synthesised de novo.

In a particular embodiment, the candidate compound according to the invention may be an antibody. The antibodies of the invention can for instance be produced by any hybridoma liable to be formed according to classical methods from splenic cells of an animal, particularly of a mouse or rat immunized against an antigenic sequence of interest. The antibodies according to this embodiment of the invention may be humanized versions of the mouse antibodies made by means of recombinant DNA technology, departing from the mouse and/or human genomic DNA sequences coding for H and L chains or from cDNA clones coding for H and L chains. Alternatively, the antibodies may be human antibodies. Such human antibodies are prepared, for instance, by means of human peripheral blood lymphocytes (PBL) repopulation of severe combined immune deficiency (SCID) mice or by using transgenic non-human animals capable of producing human antibodies. Also fragments derived from these antibodies such as Fab, F (ab)′2 ands (“single chain variable fragment”), providing they have retained the original binding properties, form part of the present invention. Such fragments are commonly generated by, for instance, enzymatic digestion of the antibodies with papain, pepsin, or other proteases. It is well known to the person skilled in the art that monoclonal antibodies or fragments thereof, can be modified for various uses. An appropriate label of the enzymatic, fluorescent, or radioactive type can label the antibodies involved in the invention. A typical antibody is an antibody newly identified as a sCD146 antibody.

In some cases, a candidate therapeutic agent has been identified and further testing may be required. In some embodiments the further testing is to evaluate a candidate therapeutic agent (or an agent that has been confirmed to be therapeutic) for quality control and/or safety concerns. In some embodiments, methods of the invention include a method of assaying a therapeutic agent (or candidate therapeutic agent) for efficacy against cancer, typically against a renal cancer, in a relevant animal model.

In a particular aspect, a method for treating a subject suffering of a renal cancer and identified (thanks to a method as herein described) as resistant to sunitinib when used as single active molecule, is herein described. The renal cancer is typically a ccRCC, in particular a metastatic ccRCC. The method typically comprises the administration of sunitinib in combination with an anti-sCD146 antibody, or the administration an anti-sCD146 antibody alone. When molecules are both administered, they are either simultaneously or sequentially (sunitinib being preferably administered first, i.e. before the anti-sCD146 antibody) administered to the subject in need thereof.

The present invention also includes kits for assessing or monitoring in vivo, in vitro or ex vivo the sensitivity or resistance of a subject having a cancer, preferably a renal cancer, to chemotherapy.

In a particular embodiment, the kit comprises detection means, possibly in suitable container means, preferably at least one, for example two, sCD146 polypeptide binding agent(s) (typically antibody(ies) specific to sCD146, typically antibody(ies) which do(es) not bind membranous CD146); optionally a molecule allowing the binding agent(s) (typically antibody(ies)) detection; and, optionally, a leaflet providing the sCD146reference expression level in a biological sample from a control or reference population or the formula for calculating the sCD146 reference expression level or threshold.

In further embodiments, the binding agent is labelled or a detection agent is included in the kit. It is contemplated that the kit may include a sCD146 polypeptide binding agent(s) attached to a non-reacting solid support, such as a tissue culture dish or a plate with multiple wells. It is further contemplated that such a kit includes at least one, for example two, detectable agent(s) in certain embodiments of the invention. In some embodiments, the invention concerns kits for carrying out a method of the invention comprising, in suitable container means: (a) an agent that specifically recognizes all or part of a sCD146 polypeptide, preferably an agent that specifically recognizes all or part of a sCD146 polypeptide; and, (b) positive control(s) that can be used to determine whether the agent is capable of specifically recognizing all or part of a sCD146 polypeptide. The kit may also include other reagents that allow visualization or other detection of the sCD146 polypeptide, such as reagents for colorimetric or enzymatic assays. Generally, the kit comprises one or more containers filled with one or more of the herein described products.

A labelling notice providing instructions for using the products in the context of a method according to the present invention can further be provided.

The present invention further relates to the use of a kit as herein described i) for assessing or monitoring in vivo, in vitro or ex vivo the sensitivity or resistance of a subject having a cancer, in particular a renal cancer, to a chemotherapy, in particular to sunitinib, and/or ii) for determining in vivo, in vitro or ex vivo the potential toxicity of said chemotherapy for a subject having a cancer, in particular a renal cancer. Preferably, the kit comprises detection means comprising at least one sCD146 binding agent and a molecule allowing the binding agent detection.

Other characteristics and advantages of the invention are given in the following experimental section (with reference to FIGS. 1 to 7), which should be regarded as illustrative and not limiting the scope of the present application. All references cited in the present application are herein incorporated by reference.

### FIGURES

FIG. 1. Variation of sCD146 plasmatic levels is indicative of PFS and OS for patients treated with sunitinib. A and B, Kaplan-Meier analysis of PFS: PFS was calculated from patient subgroups with a ratio of plasmatic levels for sCD146 between the diagnosis and after the first cycle that was less than a cut off ratio of 120%, for SUVEGIL and TORAVA trial—sunitinib group (A) or for an independent cohort—sunitinib group (B). C, Kaplan-Meier analysis of OS: OS was calculated from patient subgroups with a ratio of plasmatic levels for sCD146 between the diagnosis and after the first cycle that was less than a cut off ratio of 120%, for SUVEGIL and TORAVA trial—sunitinib group. Statistical significance (p value) and the time of PFS and OS are indicated (see Table 1).

FIG. 2. Variation of sCD146 plasmatic levels is not correlated with PFS and OS for patients treated with bevacizumab. A and B, Kaplan-Meier analysis of PFS (A) or OS (B) of patients with RCC. PFS and OS were calculated from patient subgroups with a ratio of plasmatic levels for sCD146 between the diagnosis and after the first cycle that was less than a cut off ratio of 120%, for TORAVA trial—bevacizumab group. Statistical significance (p value) and the time of PFS and OS are indicated (see Table 1).

FIG. 3. Sunitinib stimulated CD146 expression in patients treated with sunitinib in a neo-adjuvant setting. A to D, Tumors from untreated RCC patients and tumors from patients treated with sunitinib in a neo-adjuvant setting were compared (see Table 2). The levels of CD146 total (short +long form, A), CD146 long (B), CD146 short (C) and angiomotin (D) mRNA were determined by qPCR.

FIG. 4. Basal CD146 expression is higher in 786R cells and further stimulated by sunitinib. A to E, 786 and 786R cells were treated with 2.5 or 5 μM sunitinib for 48 hr. The mRNA levels of CD146 total (A), long (B) and short forms (C) and angiomotin (E) were evaluated by qPCR. D, sCD146 protein in cell supernatants was evaluated by ELISA. F, 786 cells were treated with sunitinib, in presence of recombinant sCD146 recombinant for 48 h. The cell metabolism was measured by XTT assay. Results are represented as the mean of three independent experiments±SEM.* p<0.05, ** p<0.01, *** p<0.001.

FIG. 5. Online analysis of CD146 Analysis of CD146 expression in ccRCC patients. The Kaplan-Meier analysis of progression free survival (PFS) of patients with ccRCC registered at the cbioportal database. PFS was calculated from patient subgroups with CD146 mRNA levels that were 1.5 less or greater than the median value. Statistical significance (p value) is indicated.

FIG. 6. Sunitinib but not bevacizumab (herein identified as “BVZ” or “Bev”) induces CD146 expression in RCC cells. A, 786 and 786R cells were treated with 10 μg/ml BVZ for 48 hr. sCD146 protein in cell supernatants were evaluated by ELISA. B, 786 cells were treated with 10 μg/ml BVZ, in the presence of recombinant sCD146 (rCD146) for 48 h. The cell metabolism was measured by XTT assay. C, RCC10 cells were treated with 2.5 or 5 μM sunitinib, in the presence of recombinant sCD146 (rCD146) for 48 h. The cell metabolism was measured by XTT assay. Results are represented as the mean of three independent experiments±SEM. * p<0.05, ** p<0.01, *** p<0.001.

FIG. 7. Regulation of expression of CD146 and angiomotin (Amot) is differently regulated in endothelial cells. A and B; HUVEC were treated with 2 μM sunitinib for 48 h. CD146 (A) and angiomotin (B) were analyzed by qPCR. C, HUVEC were treated with 10 μg/ml of bevacizumab (Bev), 1 or 2 μM sunitinib for 48 h. sCD146 protein in cell supernatants were evaluated by ELISA. D, HUVEC were treated with the indicated concentrations of sunitinib for 48 h. The cell metabolism was measured by XTT assay. E, HUVEC were treated with the indicated concentrations of sunitinib or Bev (10 μg/ml), in the presence of recombinant sCD146 (50 or 100 ng/ml) for 48 h. The cell metabolism was measured by XTT assay. Results are represented as the mean of three independent experiments±SEM. * p<0.05, ** p<0.01, *** p<0.001.

### EXPERIMENTAL PART

### Example 1

Soluble CD146 (sCD146) is a predictive marker of sunitinib efficacy in clear cell renal cell carcinoma (ccRCC).

### Material and Methods

### Patients

Eligible patients for SUVEGIL and TORAVA trials were at least 18 years of age and had metastatic clear cell renal cell carcinoma (ccRCC) histologically confirmed, with the presence of measurable disease according to Response Evaluation Criteria in Solid Tumors (RECIST) v1.1. Patients had not received previous systemic therapy for RCC and were eligible for sunitinib or bevacizumab combined with interferon (IFN) treatment in the first-line setting. Patients were ineligible if they had symptomatic or uncontrolled brain metastases, an estimated lifetime less than 3 months, uncontrolled hypertension or clinically significant cardiovascular events (heart failure, prolongation of the QTinterval), history of other primary cancer. All patients gave written informed consent.

TABLE 1

| COHORT | Prospective cohort | Prospective cohort | Retrospective cohort |
|---|---|---|---|
| Treatment | Stinitinib | Bevacizumab | Sunitinib |
| Number of patients | 36 | 34 | 13 |
| Gender | | | |
| Female | 6 (16.7%) | 9 (26.5%) | 3 (23.1%) |
| Male | 30 (63.3%) | 25 (73.5%) | 10 (76.9%) |
| PRIMARY TUMOR | | | |
| Fuhrman grade | | | |
| 1 | 1 (2.8%) | 2 (5.9%) | 0 (0%) |
| 2 | 9 (25%) | 8 (23.5%) | 0 (0%) |
| 3 | 17 (47.2%) | 14 (41.2%) | 6 (46.1%) |
| 4 | 9 (25%) | 10 (29.4%) | 4 (30.8%) |
| X | 0 (0%) | 0 (0%) | 3 (23.1%) |
| pT | | | |
| 1 | 8 (22.2%) | 5 (14.7%) | 1 (7.7%) |
| 2 | 10 (27.8%) | 5 (14.7%) | 1 (7.7%) |
| 3 | 16 (44.4%) | 18 (52.9%) | 8 (61.5%) |
| X | 2 (5.6%) | 6 (17.6%) | 3 (23.1%) |
| pN | | | |
| 0 | 15 (41.7%) | 0 (0%) | 4 (30.8%) |
| 1 | 5 (13.9%) | 0 (0%) | 2 (15.4%) |
| 2 | 1 (2.7%) | 0 (0%) | 1 (7.7%) |
| X | 15 (41.7%) | 34 (100%) | 6 (46.1%) |
| BEGINNING OF THE TREATMENT | | | |
| Age at therapy initiation (yr) | 60.4 (9.7) | 58.7 (11.3) | 63.5 (9.8) |
| Metastatic from the diagnosis | 18 (50%) | 14 (41.2%) | 8 (61.5%) |
| Time from diagnosis to treatment | | | |
| <1 yr | 23 (63.8%) | 20 (58.8%) | 9 (69.2%) |
| ≥1 yr | 13 (36.1%) | 14 (41.2%) | 4 (30.8%) |
| Number of metastatic sites | | | |
| 1 | 17 (47.2%) | 17 (50%) | 6 (46.1%) |
| 2 | 13 (36.1%) | 10 (29.4%) | 6 (46.1%) |
| ≥3 | 6 (16.7%) | 7 (20.6%) | 1 (7.7%) |
| Risk factor (MSKCC) | | | |
| Good | 10 (27.8%) | 14 (41.2%) | 0 (0%) |
| Intermediate | 10 (27.8%) | 18 (52.9%) | 0 (0%) |
| Bad | 8 (22.2%) | 0 (0%) | 0 (0%) |
| X | 8 (22.2%) | 2 (5.9%) | 13 (100%) |
| Median followed (month) | 34.5 (18.2-NR) | 24 (23-NR) | NA |

### Study Design (SUVEGIL and TORAVA Trial)

The prospective cohort includes patients from the SUVEGIL and TORAVA trials.

The SUVEGIL trial (clinicaltrial.gov, NCT00943839) was a multicenter prospective single-arm study. The goal of the trial is to determine whether a link exists between the effectiveness of therapy with sunitinib malate and development of blood biomarkers in patients with kidney cancer. Patients received oral sunitinib (50 mg/day) once daily for four weeks (on days 1 to 28), followed by two weeks without treatment. Courses repeat every 6 weeks in the absence of disease progression or unacceptable toxicity.

The TORAVA trial (clinicaltrial.gov, NCT00619268) was a randomized prospective study. Patient characteristics and results have been previously described (Negrier et al, 2011). Briefly, patients aged 18 years or older with untreated metastatic ccRCC were randomly assigned (2:1:1) to receive the combination of bevacizumab (10 mg/kg iv every 2 weeks) and temsirolimus (25 mg iv weekly), or the combination of interferon alfa (9 mIU iv three times per week) and bevacizumab (10 mg/kg iv every 2 weeks) (both groups are herein identified as “Bevacizumab group”), or one of the standard treatments: sunitinib (50 mg/day orally for 4 weeks followed by 2 weeks off) (Negrier et al, 2011).

### Retrospective Validation Cohort

13 patients from the HEPG (France) (50 mg/day, once daily for four weeks) were analyzed retrospectively.

### Neo-Adjuvant Patients

Samples were obtained from Nice, Bordeaux and Monaco Hospitals. Patients were treated with sunitinib (50 mg/day, once daily for four weeks) for at least two months before surgery (Table 2).

TABLE 2

| | | Stmitinib |
|---|---|---|
| GROUP | CT | neoadjuvant |
| Number of patients | 8 | 8 |
| ccRCC | 8 (100%) | 8 (100%) |
| Sex | | |
| Woman | 2 (25%) | 1 (12.5%) |
| Man | 6 (75%) | 7 (87.5%) |
| AT DIAGNOSIS | | |
| Age | 57 (42-86) | 55 (32-79) |
| Furhnnan grade | | |
| 3 | 5 (62.5%) | 5 (62.5%) |
| 4 | 3 (37.5%) | 3 (37.5%) |
| Metastatic status | | |
| M0 | 1 (12.5%) | 1 (12.5%) |
| M1 | 7 (12.5%) | 7 (87.5%) |
| Lymph node status | | |
| N0 | 6 (75%) | 5 (62.5%) |
| N1 | 2 (25%) | 3 (37.5%) |
| Number of metastasis | | |
| 0 | 1 (12.5%) | 1 (12.5%) |
| 1 | 5 (62.5%) | 4 (50%) |
| 2 | 2 (25%) | 3 (37.5%) |
| TREATMENT (NEOADJUVANT) | | |
| Duration of treatment (months) | NA | 4.4 (2-9) |

### Efficacy and Safety

Blood samples were collected during the inclusion visit (baseline) and at the end of the four weeks of sunitinib administration at each cycle for biochemical analysis.

The % variation of plasmatic level of sCD146 was correlated with OS and PFS, defined respectively as the time from inclusion in the trial to death from all causes (for OS) and to progression, treatment cessation, or death (for PFS), censored at last follow-up for those still alive or who have not progressed.

### Biochemical Analysis

Blood samples were centrifuged (10000 g for ten minutes) and the plasmas were collected and conserved at −80° C. Plasmatic level of sCD146 was determined by ELISA using a CD146 Elisa Kit (Biocytex, Marseille, France).

### Cell Culture

786-O cells (786) were purchased from the American Tissue Culture Collection. Sunitinib-resistant cells 786R were generated in the laboratory and described in (Giuliano et al, 2015).

### Gene Expression Microarray Analysis

Normalized RNA sequencing (RNA-Seq) data produced by The Cancer Genome Atlas (TCGA) were downloaded from cBiopotal(www.cbioportal.org, TCGA Provisional; RNA-Seq V2). Data were available for 503 ccRCC tumor samples. The results published here are in whole or in part based upon data generated by the TCGA Research Network: http://cancergenome.nih.gov/ (Cerami et al, 2012; Gao et al, 2013).

Quantitative Real-Time PCR (qPCR) Experiments

One microgram of total RNA was used for the reverse transcription, using the QuantiTect Reverse Transcription kit (QIAGEN, Hilden, Germany), with blend of oligo (dT) and random primers to prime first-strand synthesis. SYBR master mix plus (Eurogentec, Liege, Belgium) was used for qPCR.

### Cell Viability (XTT)

Cells (5×10³ cells/100 μl) were incubated in a 96-well plate with different effectors for the times indicated in the figure legends. Fifty microliters of sodium 3′-[1-phenylaminocarbonyl)-3,4-tetrazolium]-bis(4-methoxy-6-nitro) benzene sulfonic acid hydrate (XTT) reagent was added to each well. The assay is based on the cleavage of the yellow tetrazolium salt XTT to form an orange formazan dye by metabolically active cells. The absorbance of the formazan product, reflecting cell viability, was measured at 490 nm. Each assay was performed in quadruplicate.

### Statistical Analysis

For In Vitro Analysis

All data are expressed as the mean ±the standard error (SEM). Statistical significance and p values were determined by the two-tailed Student's t-test. Oneway ANOVA was used for statistical comparisons. Data were analyzed with Prism5.0b (GraphPad Software) by one-way ANOVA with Bonferroni post hoc.

For Patient Analysis

PFS was defined as the time between blood sample collection and progression, or death from any cause, censoring those alive and progression free at last follow-up. OS was defined as the time from blood sample collection to the date of death from any cause, censoring those alive at last follow-up.

The % variation of sCD146 between diagnosis and after 1 cycle of suntinib cut-off point was determined at 120%. Kaplan Meier method was used to produce survival curves and analyses of censored data were performed using Cox models.

### Results

Evolution of Plasmatic Levels of sCD146 is Predictive of Progression Free and Overall Survival in Metastatic RCC Patients Treated with Sunitinib

The purpose of inventors' study was to correlate sCD146 to survival. However, sCD146 levels were highly variable and they did not correlate with outcome. Hence, they analyzed sCD146 in patients that have an objective response (category 1), a stable (category 2) or a progressive disease (category 3) after the first cycle of treatment by sunitinib. Patients of categories 1 and 2 have decreased or stable levels of plasmatic sCD146 whereas patients of category 3 presented an increase in sCD146 levels (median value of up-regulation: 120%). According to the definition of this threshold, the modulation of sCD146 levels between the diagnosis and the first cycle of treatment was correlated with progression free survival (PFS). PFS was defined as the time from inclusion in the trial to progression, treatment cessation, or death, censored at last follow-up for patients that are still alive or who have not progressed.

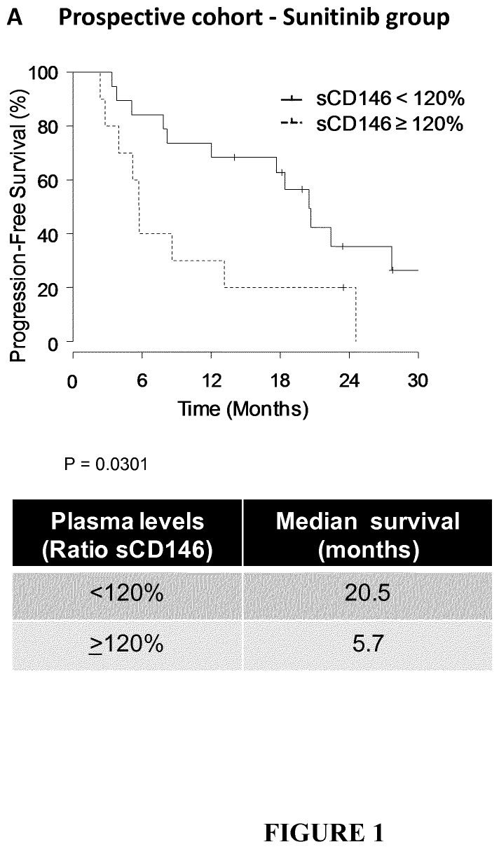

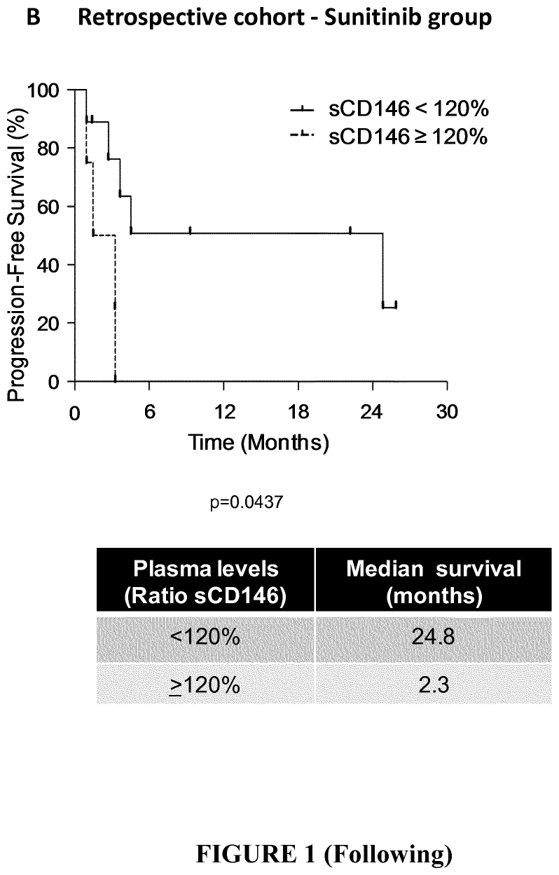

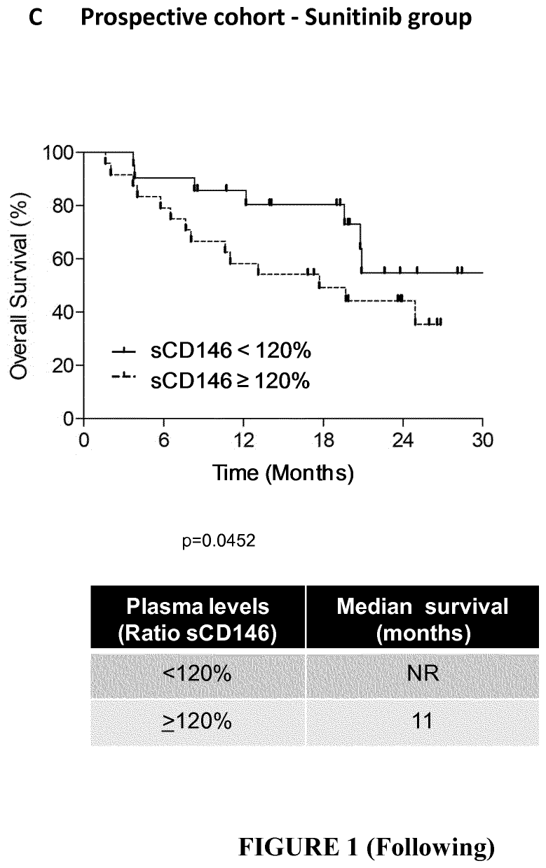

Patients included in the prospective cohort, with an increase of sCD146 plasmatic levels equal or superior to 120%, have a median PFS of 5.7 months. The PFS of the patients with a threshold increase below 120% (some of these patients have decreased or stable plasmatic sCD146 levels) was 20.5 months (p<0.0301, FIG. 1A). Equivalent results were obtained for patients of a retrospective/confirmation cohort (PFS of 24.8 months for patients with modulation of sCD146<120% and of 2.3 months for patients with sCD146≥120%, p=0.0437, FIG. 1B).

The variations of sCD146 levels (inferior, superior or equal to 120%) and clinical parameters of patients in the prospective cohort (Fuhrman grade, pT, pM or MSKCC score, the standard score used for patient evaluation in clinical practices) are not correlated.

The biological and clinical parameters were then analyzed in a multivariate Cox regression model on PFS. The variation in sCD146 levels were identified as an independent prognostic parameter for PFS (P=0.001, HR 8.298[CI 95% 2.221-31], Table 3). Therefore, the modulation of sCD145 can be considered as an independent marker for PFS.

TABLE 3

| | | Risk ratio | |
|---|---|---|---|
| Variable | Description | [IC 95% OR] | P value |
| Biological parameter | ≥120% | 1 | 0.001 |
| sCD146 | <120% | 8.298 [2.221-3] | |
| Clinical parameters | Good | 1 | 0.41 |
| MSKCC score | Intermediate | 0.542 [0.122-2.399] | 0.94 |
| | Bad | 0.955 [0.256-3.561] | |

By using the same threshold, inventors then analyzed the correlation between variations in sCD146 levels at diagnosis and after the first cycle of treatment to overall survival (OS) considered as the time from inclusion in the trial to death from all causes. The median OS of patients with sCD146<120% included in the prospective cohort was not reached (NR) whereas patients with sCD146≥120% have a median OS of 11 months (p=0.0452, FIG. 1C).

PFS and OS in a Prospective Cohort (Bevacizumab) and Correlation to Variation in sCD146 Plasmatic Levels

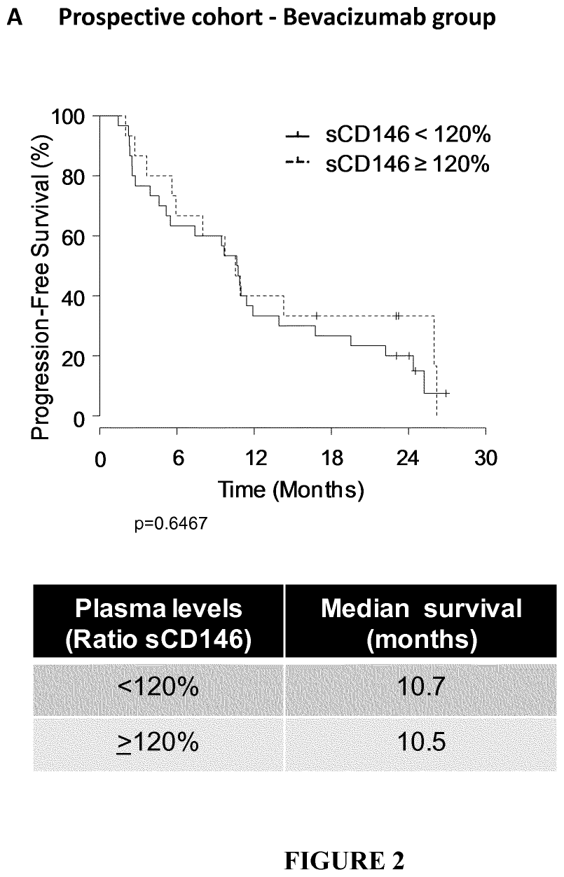

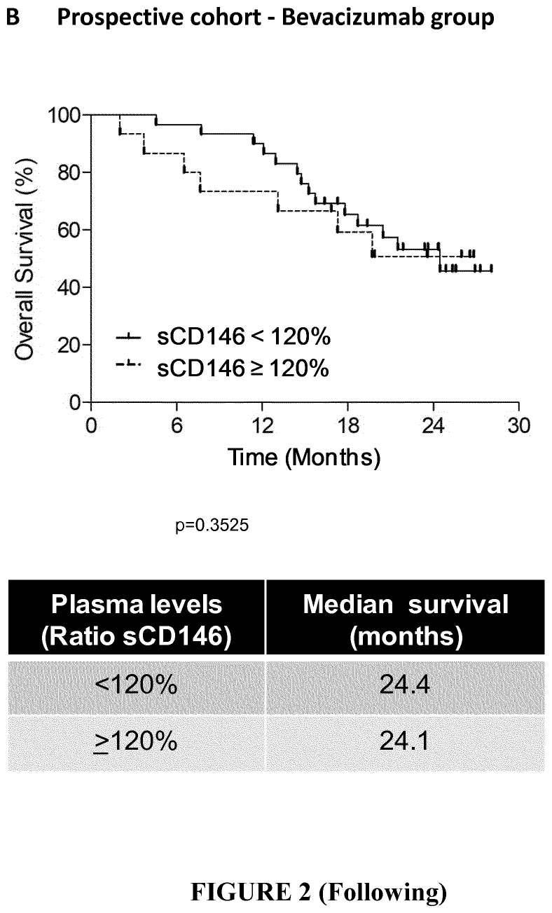

To determine the predictive role of sCD146 for sunitinib efficacy, inventors tested the modulation of sCD146 levels between the diagnosis and the first cycle of treatment in a subset of patients of the TORAVA clinical trial that were treated with bevacizumab. The difference in median PFS of patients with sCD146<120% included in the prospective cohort (10.7 months) and of patients with sCD146≥120% (10.5 months) was not significant (p=0.6467, FIG. 2A). An equivalent trend was obtained for OS (median 24.4 months for patients with sCD146<120% and 24.1 months for patients with sCD146≥120%, p=0.3525, FIG. 2B). These results demonstrate that the 120% increase in sCD146 levels are predictive of sunitinib but not of bevacizumab efficacy.

Sunitinib Stimulates CD146 mRNA Expression in Patients Treated with Sunitinib

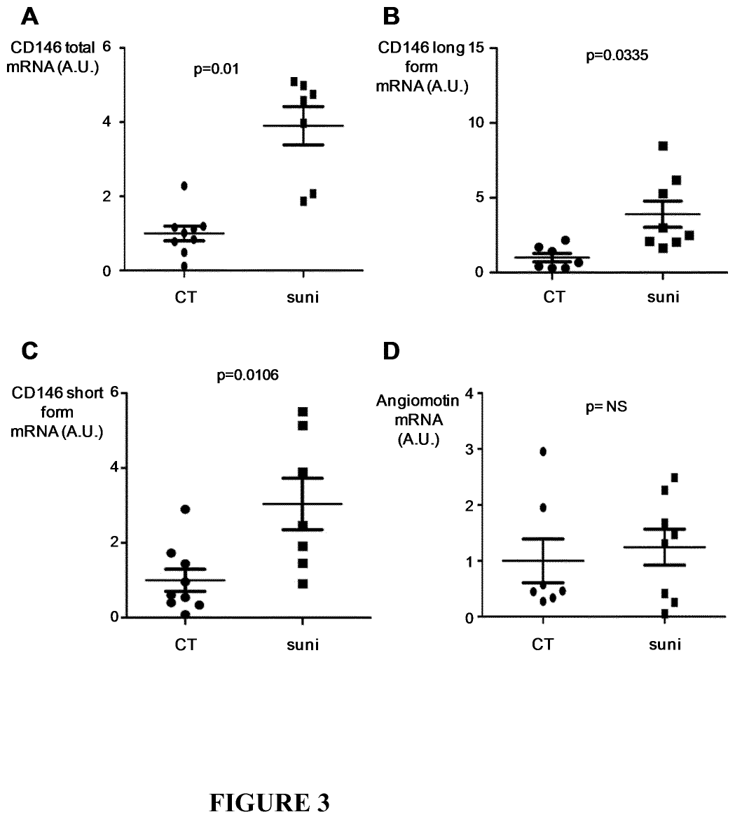

Inventors analyzed the expression of CD146 and angiomotin (Amot) mRNA in tumors from patients treated with sunitinib in a neo-adjuvant setting as compared to tumors from untreated RCC patients (FIG. 3). Results show that total CD146 mRNA was significantly increased in sunitinib-treated patients (FIG. 3A). This increase was observed for both the long (FIG. 3B) and short (FIG. 3C) isoforms of CD146. In contrast, no modification of angiomotin mRNA expression was observed between the two groups (FIG. 3D).

Sunitinib Stimulates CD146 Expression and sCD146 Production in Sunitinib-Resistant RCC Cells

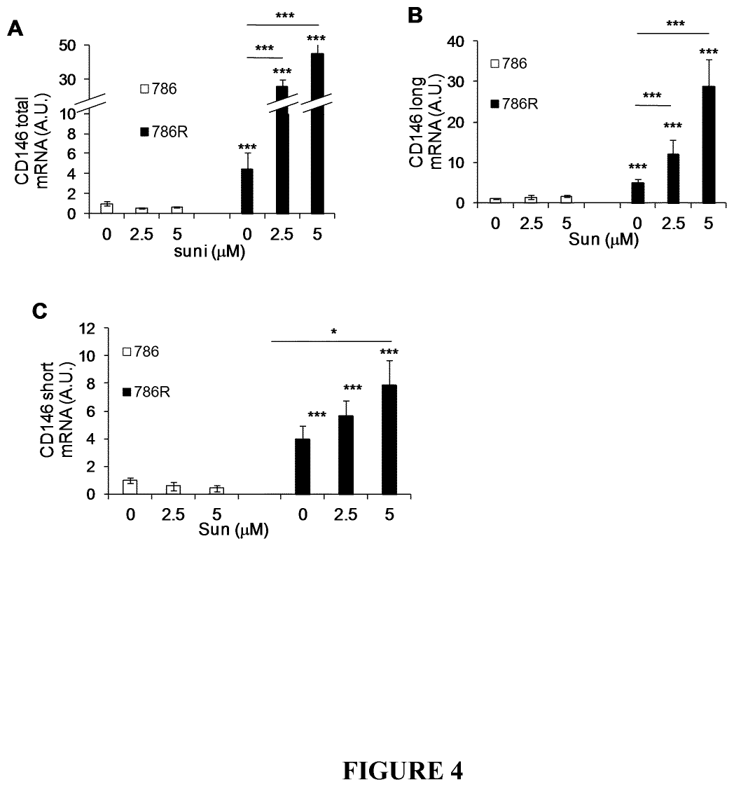

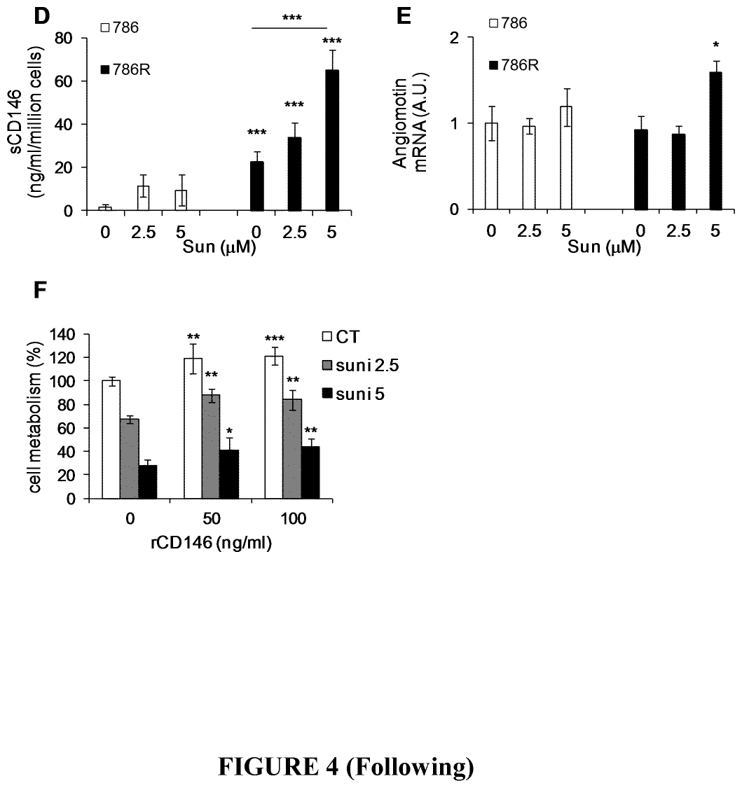

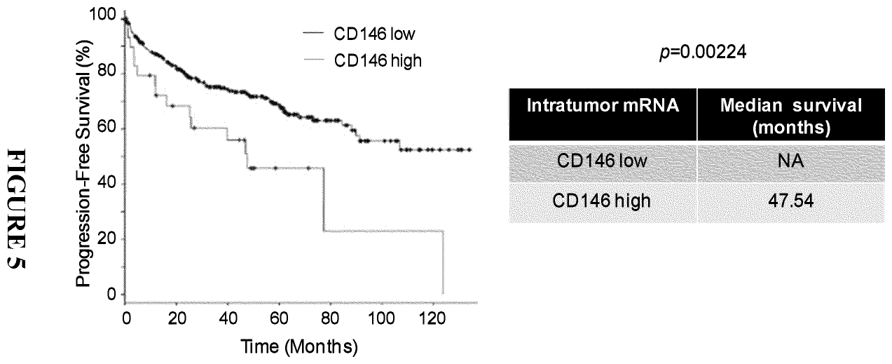

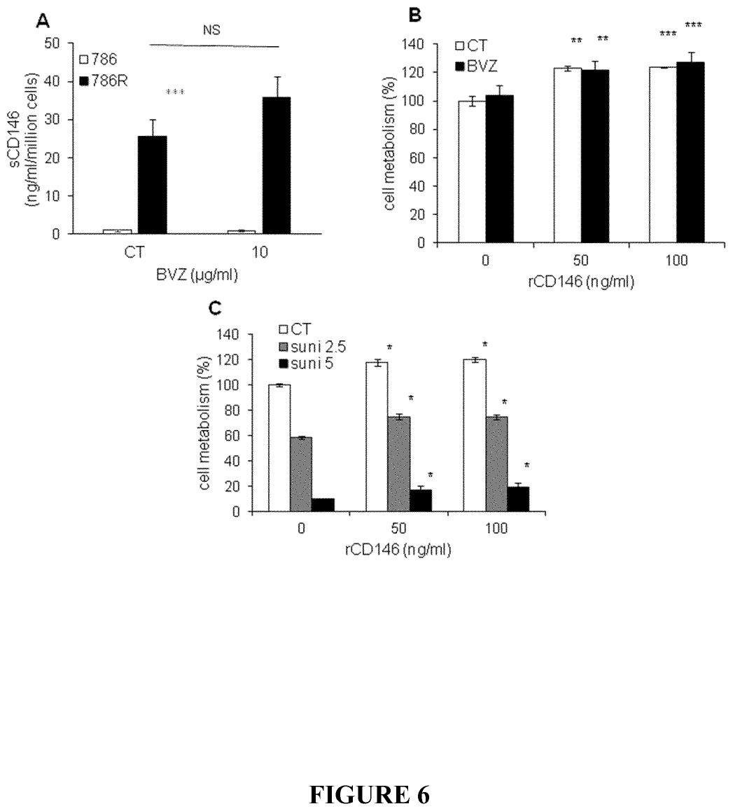

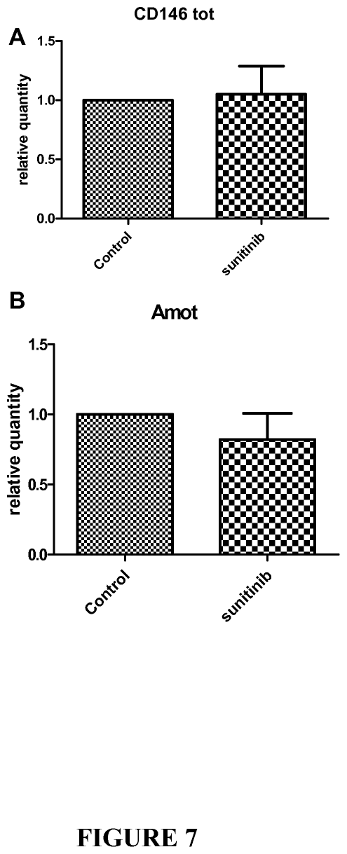

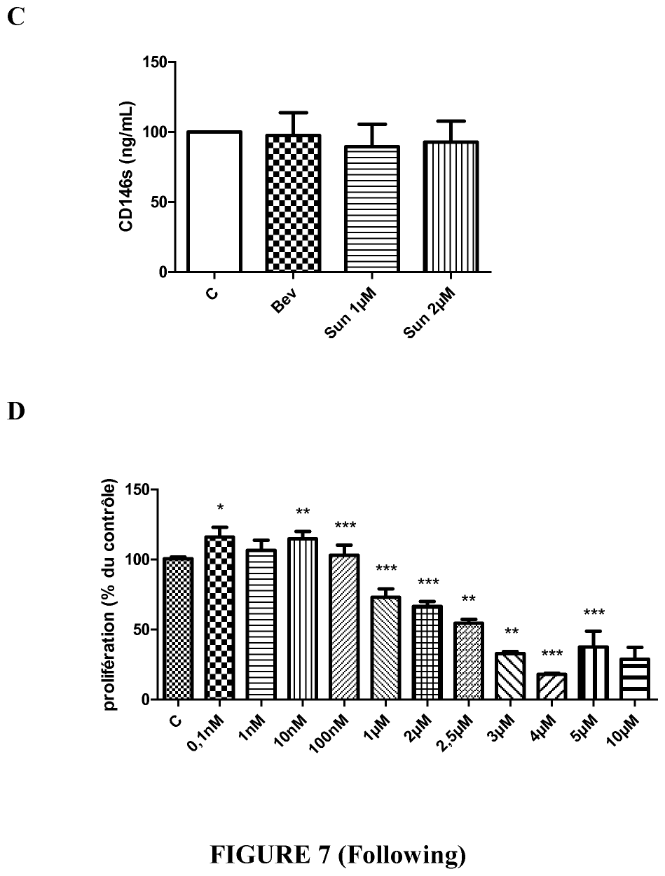

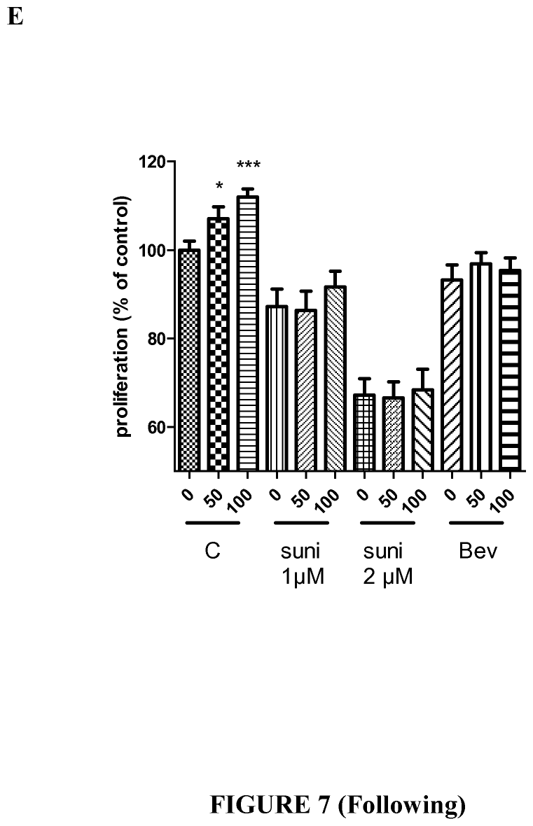

To confirm these results, inventors used RCC cells which are respectively sensitive (786) and resistant (786R) cells to sunitinib. Cells were treated or not with 2.5 and 5 μM sunitinib for 48 h. Total CD146 mRNA expression increased in a dose-dependent manner by sunitinib in 786R but not in 786 cells (FIG. 4A). This increase was observed for the long (FIG. 4B) and short (FIG. 4C) CD146 mRNA. This increase did not occur in HUVEC treated with sunitinib, at least at the mRNA level (FIG. 7A). Of interest, the increase in membrane CD146 observed in 786R cells after sunitinib treatment was accompanied by an increase in sCD146 production (FIG. 4D). Concerning angiomotin mRNA expression, it was only modified when 786R cells were treated with 5 μM sunitinib (FIG. 4E). In HUVEC, there was no modification (FIG. 7B). Even in the absence of sunitinib treatment, sCD146 secretion was higher in 786R cells than in 786 cells. In contrast, there was no increase in sCD146 secretion when 786R cells were treated with bevacizumab (FIG. 6A) or when HUVEC cells were treated with 1 or 2 μM of sunitinib (FIG. 7C). In experiments with HUVEC, these concentrations of sunitinib were used because they promoted about 50% decrease in cell proliferation (FIG. 7D).

Inventors then analyzed the effect of two concentrations of sCD146 (50 and 100 ng/ml) on the metabolism of 786 cells treated with 2.5 or 5 μM sunitinib. sCD146 increased cell metabolism at both concentrations. Moreover, it also prevents sunitinib-dependent decreased cell metabolism (FIG. 4F). In contrast, whereas sCD146 stimulated the proliferation of HUVEC, sCD146 did not protect HUVEC from sunitinib (FIG. 7E).

### Discussion

No curative treatment exists for RCC. Therefore, it remains a disease with poor prognosis. Two types of patients exist; i) M0 patients that have a good prognosis although some patients progress toward M1 phase without any robust method to predict such pejorative evolution; ii) M1 patients that are currently treated with sunitinib. The efficacy in tumor control varies from few days to several years (Gore et al, 2015). Unfortunately, relapses are synonymous of ineluctable deaths in a few months delay. However, RCC was probably the only disease that led to the development of more than ten treatments these last years. Therefore, the identification of predictive marker(s) of M0/M1 evolution and of sunitinib efficacy may allow a more accurate survey of at risk patients and a rapid switch to a second line of treatment before the observation of relapse by CT scans. Such markers must be detectable; i) in material sampled in patients by a non-invasive technique (urine, blood, typically serum and plasma); ii) by a simple and sensitive method easily transposable in clinical practices. The presence of high mRNA amounts in the tumor of patients that relapsed toward a M1 phenotype are preferably to be confirmed for example in plasma of MO patients. Plasmatic sCD146 with an increase superior or equal to 120% between the diagnosis and the first cycle of sunitinib represents such relevant marker of relapse. This simple detection predicts pejorative evolution that may occur after several cycles of treatment. This detection highlights a relapse earlier than those detected by CT scans that are generally performed after the second cycle of sunitinib. sCD146 is generated by the shedding of the membrane form of CD146 through a yet unidentified process that might involve matrix metalloproteases (Bardinet al, 2009). CD146 is expressed on endothelial and several cancer cells including RCC cells. In tumors, inventors observed a large increase in the long and short forms of CD146 mRNA which may be related to an up-regulation on tumor and/or tumor-associated endothelial cells. The role of both isoforms in cancer cells is unknown. However, inventors have shown that the long CD146 isoform is essentially involved in structural functions at the junction, whereas short CD146 isoform is involved in angiogenic functions in endothelial cells. Further studies will be necessary to delineate the roles of these forms in RCC cells. To discriminate between effects on cancer and endothelial cells, inventors performed in vitro experiments with HUVEC and 786 and 786R cells. Whereas no effect of sunitinib was observed on CD146 mRNA expression in HUVEC and a decreased expression in 786 cells, a huge increase of total, long and short forms was observed in 786R cells. This effect on mRNA was associated with an increase in sCD146 production, in cell metabolism and in the number of viable 786R cells. These results were also consistent with increased plasmatic levels equal to or above the threshold of 120 percent observed in patients that relapsed on sunitinib. Thus, these experiments strongly suggest that one of the mechanisms of resistance to sunitinib may depend on a huge increase in CD146/sCD146 expression.

### Conclusion

Inventors' study defines soluble CD146 as a relevant early predictive marker of response to sunitinib in RCC. They established that an increase ≥120% of sCD146 in patients after sunitinib treatment is indicative of a high risk of relapse in prospective and retrospective independent cohorts. Based on these results, inventors suggest introducing the sCD146 measurement into the clinical practices to rapidly adapt the treatment of patients identified as resistant to sunitinib thanks to the present invention.

In addition, inventors' study reveals the interest of targetingsCD146 with a sCD146 therapeutic antibody, preferably along with sunitinib, in patients with RCC identified as resistant to sunitinib when used as single active drug in the treatment of cancer, in particular in the first line of treatment of renal cancer.

### REFERENCES

Bardin N, Anfosso F, Masse J M, Cramer E, Sabatier F, Le Bivic A, Sampol J, Dignat-George F (2001) Identification of CD146 as a component of the endothelial junction involved in the control of cell-cell cohesion. Blood 98: 3677-3684

Bardin N, Blot-Chabaud M, Despoix N, Kebir A, Harhouri K, Arsanto J P, Espinosa L, Perrin P, Robert S, Vely F, Sabatier F, Le Bivic A, Kaplanski G, Sampol J, Dignat-George F (2009) CD146 and its soluble form regulate monocyte transendothelial migration. Arterioscler Thromb Vasc Biol 29: 746-753

Bardin N, Frances V, Combes V, Sampol J, Dignat-George F (1998) CD146: biosynthesis and production of a soluble form in human cultured endothelial cells. FEBS Lett 421: 12-14

Bardin N, Moal V, Anfosso F, Daniel L, Brunet P, Sampol J, Dignat George F (2003) Soluble CD146, a novel endothelial marker, is increased in physiopathological settings linked to endothelial junctional alteration. Thromb Haemost 90: 915-920

Cerami E, Gao J, Dogrusoz U, Gross B E, Sumer S O, Aksoy B A, Jacobsen A, Byrne C J, Heuer M L, Larsson E, Antipin Y, Reva B, Goldberg A P, Sander C, Schultz N (2012) The cBio cancer genomics portal: an open platform for exploring multidimensional cancer genomics data. Cancer Discov 2: 401-404

Choueiri T K, Escudier B, Powles T, Mainwaring P N, Rini B I, Donskov F, Hammers H, Hutson T E, Lee J L, Peltola K, Roth B J, Bjarnason G A, Geczi L, Keam B, Maroto P, Heng D Y, Schmidinger M, Kantoff P W, Borgman-Hagey A, Hessel C et al (2015) Cabozantinib versus Everolimus in Advanced Renal-Cell Carcinoma. N Engl J Med 373: 1814-1823

Choueiri T K, Plantade A, Elson P, Negrier S, Ravaud A, Oudard S, Zhou M, Rini B I, Bukowski R M, Escudier B (2008) Efficacy of sunitinib and sorafenib in metastatic papillary and chromophobe renal cell carcinoma. J Clin Oncol 26: 127-131

Escudier B, Albiges L, Sonpavde G (2013) Optimal management of metastatic renal cell carcinoma: current status. Drugs 73: 427-438

Escudier B, Sharma P, McDermott D F, George S, Hammers H J, Srinivas S, Tykodi S S, Sosman J A, Procopio G, Plimack E R, Castellano D, Gurney H, Donskov F, Peltola K, Wagstaff J, Gauler T C, Ueda T, Zhao H, Waxman I M, Motzer R J (2017) CheckMate 025 Randomized Phase 3 Study: Outcomes by Key Baseline Factors and Prior Therapy for Nivolumab Versus Everolimus in Advanced Renal Cell Carcinoma. Eur Urol

Feng G, Fang F, Liu C, Zhang F, Huang H, Pu C (2012) CD146 gene expression in clear cell renal cell carcinoma: a potential marker for prediction of early recurrence after nephrectomy. Int Urol Nephrol 44: 1663-1669

Gao J, Aksoy B A, Dogrusoz U, Dresdner G, Gross B, Sumer S O, Sun Y, Jacobsen A, Sinha R, Larsson E, Cerami E, Sander C, Schultz N (2013) Integrative analysis of complex cancer genomics and clinical profiles using the cBioPortal. Sci Signal 6: p11

Giuliano S, Cormerais Y, Dufies M, Grepin R, Colosetti P, Belaid A, Parola J, Martin A, Lacas-Gervais S, Mazure N M, Benhida R, Auberger P, Mograbi B, Pages G (2015) Resistance to sunitinib in renal clear cell carcinoma results from sequestration in lysosomes and inhibition of the autophagic flux. Autophagy 11: 1891-1904

Gore M E, Szczylik C, Porta C, Bracarda S, Bjarnason G A, Oudard S, Lee S H, Haanen J, Castellano D, Vrdoljak E, Schoffski P, Mainwaring P, Hawkins R E, Crino L, Kim T M, Carteni G, Eberhardt W E, Zhang K, Fly K, Matczak E et al (2015) Final results from the large sunitinib global expanded-access trial in metastatic renal cell carcinoma. Br J Cancer 113: 12-19

Harhouri K, Kebir A, Guillet B, Foucault-Bertaud A, Voytenko S, Piercecchi-Marti M D, Berenguer C, Lamy E, Vely F, Pisano P, Ouafik L, Sabatier F, Sampol J, Bardin N, Dignat-George F, Blot-Chabaud M (2010) Soluble CD146 displays angiogenic properties and promotes neovascularization in experimental hind-limb ischemia. Blood 115: 3843-3851

Jiang T, Zhuang J, Duan H, Luo Y, Zeng Q, Fan K, Yan H, Lu D, Ye Z, Hao J, Feng J, Yang D, Yan X (2012) CD146 is a coreceptor for VEGFR-2 in tumor angiogenesis. Blood 120: 2330-2339

Lehmann J M, Riethmuller G, Johnson J P (1989) MUC18, a marker of tumor progression in human melanoma, shows sequence similarity to the neural cell adhesion molecules of the immunoglobulin superfamily. Proc Natl Acad Sci USA 86: 9891-9895

Liu W F, Ji S R, Sun J J, Zhang Y, Liu Z Y, Liang A B, Zeng H Z (2012) CD146 expression correlates with epithelial-mesenchymal transition markers and a poor prognosis in gastric cancer. Int J Mol Sci 13: 6399-6406

Maitra A, Hansel D E, Argani P, Ashfaq R, Rahman A, Naji A, Deng S, Geradts J, Hawthorne L, House M G, Yeo C J (2003) Global expression analysis of well-differentiated pancreatic endocrine neoplasms using oligonucleotide microarrays. Clin Cancer Res 9: 5988-5995

Motzer R J, Escudier B, Oudard S, Hutson T E, Porta C, Bracarda S, Grunwald V, Thompson J A, Figlin R A, Hollaender N, Urbanowitz G, Berg W J, Kay A, Lebwohl D, Ravaud A (2008) Efficacy of everolimus in advanced renal cell carcinoma: a double-blind, randomised, placebo-controlled phase III trial. Lancet 372: 449-456

Motzer R J, Escudier B, Tomczak P, Hutson T E, Michaelson M D, Negrier S, Oudard S, Gore M E, Tarazi J, Hariharan S, Chen C, Rosbrook B, Kim S, Rini B I (2013a) Axitinib versus sorafenib as second-line treatment for advanced renal cell carcinoma: overall survival analysis and updated results from a randomised phase 3 trial. Lancet Onco 114: 552-562

Motzer R J, Hutson T E, Cella D, Reeves J, Hawkins R, Guo J, Nathan P, Staehler M, de Souza P, Merchan J R, Boleti E, Fife K, Jin J, Jones R, Uemura H, De Giorgi U, Harmenberg U, Wang J, Sternberg C N, Deen K et al (2013b) Pazopanib versus sunitinib in metastatic renal-cell carcinoma. N Engl J Med 369: 722-731

Motzer R J, Hutson T E, Ren M, Dutcus C, Larkin J (2016) Independent assessment of lenvatinib plus everolimus in patients with metastatic renal cell carcinoma. Lancet Oncol 17: e4-5

Mulders P F (2008) Renal cell carcinoma. World J Urol 26: 113

Negrier S, Gravis G, Perol D, Chevreau C, Delva R, Bay J O, Blanc E, Ferlay C, Geoffrois L, Rolland F, Legouffe E, Sevin E, Laguerre B, Escudier B (2011) Temsirolimus and bevacizumab, or sunitinib, or interferon alfa and bevacizumab for patients with advanced renal cell carcinoma (TORAVA): a randomised phase 2 trial. Lancet Oncol 12: 673-680

Oka S, Uramoto H, Chikaishi Y, Tanaka F (2012) The expression of CD146 predicts a poor overall survival in patients with adenocarcinoma of the lung. Anticancer Res 32: 861-864

Roudier E, Chapados N, Decary S, Gineste C, Le Bel C, Lavoie J M, Bergeron R, Birot O (2009) Angiomotin p80/p130 ratio: a new indicator of exercise-induced angiogenic activity in skeletal muscles from obese and non-obese rats? J Physiol 587: 4105-4119

Satchi-Fainaro R, Ferber S, Segal E, Ma L, Dixit N, Ijaz A, Hlatky L, Abdollahi A, Almog N (2012) Prospective identification of glioblastoma cells generating dormant tumors. PLoS One7: e44395

Stalin J, Harhouri K, Hubert L, Subrini C, Lafitte D, Lissitzky J C, Elganfoud N, Robert S, Foucault-Bertaud A, Kaspi E, Sabatier F, Aurrand-Lions M, Bardin N, Holmgren L, Dignat-George F, Blot-Chabaud M (2013) Soluble melanoma cell adhesion molecule (sMCAM/sCD146) promotes angiogenic effects on endothelial progenitor cells through angiomotin. J Biol Chem 288: 8991-9000

Stalin J, Nollet M, Garigue P, Fernandez S, Vivancos L, Essaadi A, Muller A, Bachelier R, Foucault-Bertaud A, Fugazza L, Leroyer AS, Bardin N, Guillet B, Dignat-George F, Blot-Chabaud M (2016) Targeting soluble CD146 with a neutralizing antibody inhibits vascularization, growth and survival of CD146-positive tumors. Oncogene 35: 5489-5500

Wu G J, Fu P, Chiang C F, Huss W J, Greenberg N M, Wu M W (2005) Increased expression of MUC18 correlates with the metastatic progression of mouse prostate adenocarcinoma in the TRAMP model. J Urol 173: 1778-1783

Zeng G F, Cai S X, Wu G J (2011) Up-regulation of METCAM/MUC18 promotes motility, invasion, and tumorigenesis of human breast cancer cells. BMC Cancer 11: 113

Zhao B, Li L, Lu Q, Wang L H, Liu C Y, Lei Q, Guan K L (2011) Angiomotin is a novel Hippo pathway component that inhibits YAP oncoprotein. Genes Dev 25: 51-63

1-9. (canceled)

10. An in vitro or ex vivo method of assessing the sensitivity of a subject having a renal cancer to sunitinib for treating renal cancer, which method comprises a step of determining, in a biological sample from said subject, the expression level of soluble CD146 (sCD146) and a step of comparing said sCD146 expression level to a sCD146 reference expression level, thereby assessing whether the subject having renal cancer is sensitive or resistant to sunitinib.

11. The method according to claim 10, wherein the sCD146 expression level is determined after treatment of the subject with sunitinib, and the sCD146 reference expression level is calculated from the sCD146 expression level determined in a biological sample from said subject before administration of sunitinib to said subject.

12. The method according to claim 10, wherein the biological sample is a plasma or serum sample or a derivative thereof.

13. The method according to claim 10, wherein the sCD146 reference expression level is the sCD146 expression level determined in a biological sample from the subject before administration of sunitinib to said subject to which is added (plus) 120% of said sCD146 expression level determined in the biological sample from the subject before administration of sunitinib.

14. The method according to claim 10, wherein a sCD146 expression level below the sCD146 reference expression level is indicative of a sensitivity of the subject to sunitinib, and wherein a sCD146 expression level identical to or above said reference expression level is indicative of a resistance of the subject to sunitinib.

15. The method according to claim 10, wherein the subject is a subject who has undergone at least partial resection of the cancerous tumor.

16. The method according to claim 10, wherein the renal cancer is a metastatic clear cell renal cancer (ccRCC).

17. An in vitro or ex vivo method of selecting, for a subject having a renal cancer, a treatment efficient against renal cancer in the subject, wherein the method comprises a step a) of determining the sCD146 expression level in a biological sample of the subject having renal cancer before administration to the subject of sunitinib, and a step b) of comparing said sCD146 expression level to sCD146 reference expression level, a sCD146 expression level identical to or above the sCD146 reference expression level, being the indication that sunitinib will not be efficient, or will not be efficient alone in the subject, and a step c) of selecting an appropriate treatment of renal cancer in the subject, for example combining sunitinib with an additional compound such as a sCD146 antibody; and, on the contrary, a sCD146 expression below the sCD146 reference expression level being the indication that sunitinib will be efficient alone against renal cancer in the subject.


SEQUENCE LISTING (16 sequences)

SEQ ID NO: 1 - protein, 646 aa, Homo sapiens
  MISC_FEATURE: Amino acid sequence of human long CD146 protein
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKTELVVEVKSDKLPEEMGLLQGSSGDKRAPGDQGEKYIDLRH

SEQ ID NO: 2 - protein, 604 aa, Homo sapiens
  MISC_FEATURE: Amino acid sequence of human short CD146 protein
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEMERNTSI

SEQ ID NO: 3 - protein, 552 aa, Homo sapiens
  MISC_FEATURE: Amino acid sequence of human soluble CD146 protein
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKL

SEQ ID NO: 4 - protein, 553 aa, Homo sapiens
  MISC_FEATURE: Amino acid sequence of human soluble CD146 protein
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLP

SEQ ID NO: 5 - protein, 554 aa, Homo sapiens
  MISC_FEATURE: Amino acid sequence of human soluble CD146 protein
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPE

SEQ ID NO: 6 - protein, 555 aa, Homo sapiens
  MISC_FEATURE: Amino acid sequence of human soluble CD146 protein
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEP

SEQ ID NO: 7 - protein, 556 aa, Homo sapiens
  MISC_FEATURE: Amino acid sequence of human soluble CD146 protein
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPE

SEQ ID NO: 8 - protein, 557 aa, Homo sapiens
  MISC_FEATURE: Amino acid sequence of human soluble CD146 protein
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPES

SEQ ID NO: 9 - protein, 558 aa, Homo sapiens
  MISC_FEATURE: Amino acid sequence of human soluble CD146 protein
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESR

SEQ ID NO: 10 - DNA, 1656 bp, Homo sapiens
  misc_feature: Nucleic acid sequence of human soluble CD146 protein
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctg

SEQ ID NO: 11 - DNA, 1659 bp, Homo sapiens
  misc_feature: Nucleic acid sequence of human soluble CD146 protein
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccg

SEQ ID NO: 12 - DNA, 1662 bp, Homo sapiens
  misc_feature: Nucleic acid sequence of human soluble CD146 protein
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggag

SEQ ID NO: 13 - DNA, 1665 bp, Homo sapiens
  misc_feature: Nucleic acid sequence of human soluble CD146 protein
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccg

SEQ ID NO: 14 - DNA, 1668 bp, Homo sapiens
  misc_feature: Nucleic acid sequence of human soluble CD146 protein
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggag

SEQ ID NO: 15 - DNA, 1671 bp, Homo sapiens
  misc_feature: Nucleic acid sequence of human soluble CD146 protein
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggagagc

SEQ ID NO: 16 - DNA, 1674 bp, Homo sapiens
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggagagccgg